(19) United States
(12) Patent Application Publication
(10) Pub. No.: US 2003/0027231 A1
(43) Pub. Date: Feb. 6, 2003

(54) Methods for using mass spectrometry to identify and classify filamentous fungi, yeasts, molds and pollen

(76) Inventors: Wayne A. Bryden, Ellicott City, MD (US); Marisa A. Lalekos, Olney, MD (US); Johns S. Morgan, Laurel, MD (US)

Correspondence Address: Francis A Cooch, The Johns Hopkins University, Applied Physic Laboratory, 11100 Johns Hopkins Road, Laurel, MD, 20723-6099, US

(21) Appl. No.: 10/031,036

(22) PCT Filed: May 23, 2001
(86) PCT No.: PCT/US01/16696

(51) Int. Cl. G01N 33/53; G01N 33/569; C12Q 1/04

(52) U.S. Cl. 435/7.31; 435/34

(57) ABSTRACT

A method for the identification and classification of filamentous fungi, yeasts, molds, toxins produced by fungi, and pollen in air environmental and biological samples using genus, species and strain specific biomarkers is provided. The biomarkers, can be generated using mass spectrometers and particularly by matrix assisted laser desorption ionization time-of-flight mass spectrometry (MAILDI-TOF-MS) analyses of filamentous fungi, yeasts, molds, toxins produced by fungi, and pollen.

### CROSS-REFERENCE TO RELATED APPLICATIONS

This application claims priority to U.S. Provisional Application 60/207,964, filed May 31, 2000, entitled “Methods for Using Mass Spectrometry to Identify Pollens and Identify and Classify Filamentous Fungi, Yeast and Molds” of Bryden et al. The contents of the aforesaid U.S. Provisional Application No. 60/207,964 are hereby incorporated by reference.

### BACKGROUND OF THE INVENTION

1. Field of the Invention

The present disclosure relates generally to the identification and classification of filamentous fungi, yeasts, molds, toxins produced by fungi, and pollen in environmental and biological samples. More particularly, the present disclosure is directed to a method for the identification and classification of filamentous fungi, yeasts, molds, toxins produced by fungi, and pollen with genus, species and strain specific biomarkers generated by mass spectrometry.

2. Description of the Related Art

Efforts are underway to develop systems to rapidly and accurately identify microbes which is critical in diagnosing diseases, predicting on-coming public health hazards, monitoring potential contamination in stored foods and grains, regulating bioprocessing operations and particularly in view of the threat posed by biological and chemical weapons of mass destruction. Mass spectrometers have been employed in identifying such microbes. Mass spectrometers are commercially available and can include, for example, single or multiple quadrupole, single or multiple magnetic sector, Fourier Transform ion cyclotron resonance (FTICR), ion trap, and combinations thereof (e.g., ion-trap/time-of-flight).

Generally, mass spectrometry is an analytical technique used for the accurate determination of molecular weights, the identification of chemical structures, the determination of the compositions of mixtures, and qualitative and/or quantitative elemental analysis. In operation, a mass spectrometer generates ions of sample molecules under investigation, separates the ions according to their mass-to-charge ratio (m/z), and measures the relative abundance of each ion. This analysis of the mass distribution of the molecule and its ion fragments can lead to a molecular “fingerprint” for identification.

One type of mass spectrometer is a time-of-flight (TOF) mass spectrometer which separates ions according to their mass-to-charge ratio by measuring the time it takes generated ions to travel to a detector. See, e.g., U.S. Pat. Nos. 5,045,694 and 5,160,840, the contents of which are incorporated herein by reference. TOF mass spectrometers are advantageous because they are relatively simple instruments with virtually unlimited mass-to-charge ratio range. TOF mass spectrometers have potentially higher sensitivity than traditional scanning instruments because they can record all the ions generated from each ionization event TOF mass spectrometers are particularly useful for measuring the mass-to-charge ratio of large organic molecules where conventional magnetic field mass spectrometers lack sensitivity.

Typically, TOF mass spectrometers include an ionization source for generating ions of sample material under investigation. The ionization source contains one or more electrodes or electrostatic lenses for accelerating and properly directing the ion beam. In the simplest case the electrodes are grids. A detector is positioned at a predetermined distance from the final grid for detecting ions as a function of time. Generally, a drift region exists between the final grid and the detector. The drift region allows the ions to travel, in free flight, a predetermined distance before they impact the detector.

The flight time of an ion accelerated by a given electric potential is proportional to its mass-to-charge ratio. Thus, the time-of-flight of an ion is a function of its mass-to-charge ratio, and is approximately proportional to the square root of the mass-to-charge ratio. Assuming the presence of only singly charged ions, the lightest group of ions reaches the detector first and is followed by groups of successively heavier mass groups. In practice, however, ions of equal mass and charge do not arrive at the detector at exactly the same time. This occurs primarily because of the initial temporal, spatial, and kinetic energy distributions of generated ions. These initial distributions lead to broadening of the mass spectral peaks thus limiting the resolving power of TOF spectrometers. The ion source plays a major role in obtaining distinct identifiable peaks.

One example of an ionization source frequently used in mass spectrometry is Electron Ionization (EI). In EI, the sample is vaporized within the mass spectrometer prior to passing into an electron ionization region, where it is subjected to an electron beam. While databases have been created that contain the EI mass spectra of over 100,000 compounds, facilitating the structural determination of molecules, EI is limited in its application to small molecules below the range of common bioorganic compounds. (see, e.g., Siuzdak, Mass Spectrometry for Biotechnology, pp. 67 (1996).) EI is inadequate in treating compounds above a molecular weight of about 400 Da because the thermal treatment causes decomposition of the analyte to be tested prior to vaporization and can lead, in many cases, to excessive fragmentation.

Yet other ionization techniques used in mass spectrometry are laser desorption (LD) and other “soft” ionization techniques, such as fast atom bombardment (FAB), plasma desorption and electrospray ionization (ESI). These techniques were developed to address the problem of ionizing polar, thermally labile, nonvolatile compounds, such as bioorganic molecules, for mass spectrometric analysis. These properties are typical of bioorganic molecules (e.g., proteins, nucleic acids, oligosaccharides) and preclude or interfere with the acquisition of their spectra using a “Iard” ionization technique such as electron impact.

In FAB, the sample to be analyzed is added to a matrix, usually a nonvolatile solvent in which the sample is dissolved, prior to analysis. FAB typically uses a direct insertion probe for sample introduction and a high-energy beam of Xe atoms, Cs⁺ ions, or NH₄⁺ clusters to sputter the sample and matrix from the probe surface. The matrix replenishes the surface with new sample as the ion beam bombards the surface and absorbs most of the incident energy thus minimizing damage to the sample from the high-energy particle beam. The matrix is also believed to facilitate the ionization process. The two most common matrices used are m-nitrobenzyl alcohol and glycerol. The ion beam desorbs the sample of interest from the matrix solution into the gas phase; charged molecules are then electrostatically propelled into the mass analyzer. Massive Cluster Impact (MCI), a form of FAB known to those skilled in the art, produces multiply charged ions making it more suitable for use with high-molecular weight biopolymers.

ESI is used to produce gaseous ionized molecules from a liquid solution containing the analyte. A fine spray of highly charged droplets is created in the presence of a strong electric field at the tip of a metal nozzle maintained at about 4000 V, which are then attracted to the mass spectrometer inlet Dry gas, heat, or both arc applied to the droplets before they enter the mass spectrometer so that the solvent evaporates from the surface. The electric field density on the surface of the droplet increases as its size decreases, eventually leading to the expulsion of ions, which are directed to an orifice leading to the mass analyzer. ESI is conducive to the formation of multiply charged molecules, making it possible to obtain spectra from high molecular weight compounds.

Of all the desorption techniques, plasma desorption and laser desorption result in the smallest initial spatial distributions, and thus less broadening of mass spectral peaks, because ions originate from well defined areas on the sample surface and the initial spatial uncertainty of ion formation is negligible. These methods, especially laser ionization methods, make it possible to preserve useful structural information and directly obtain mass spectra from polar, high molecular weight compounds encountered in microbiological studies without resorting to pyrolysis and other more destructive techniques.

Laser desorption is considered a soft ionization technique because the resulting spectra are dominated by molecular ions instead of fragment ions. Conventional LD typically employs sufficiently short pulses (frequently less than 10 nanoseconds) to minimize temporal uncertainty. However, in some cases, ion generation may continue for some time after the laser pulse terminates causing loss of resolution due to temporal uncertainty. The main requirement, and perhaps the main drawbacks of LD/MS is that analytes must absorb at the wavelength emitted by the laser. These requirements limit the range of compounds studied using LD/MS.

The performance of LD maybe substantially improved by the addition of a small organic matrix molecule to the sample material. This technique, known as matrix-assisted laser desorption/ionization MLD), is based on the discovery that the desorption/ionization of large, nonvolatile molecules such as proteins can be effected when a sample of such molecules is irradiated after being codeposited with a large molar excess of an energy-absorbing “matrix” material, even though the molecule does not strongly absorb at the wavelength of the laser radiation. The abrupt energy absorption initiates a phase change in a microvolume of the absorbing sample from a solid to a gas while also inducing ionization of the sample molecules. MALDI is particularly advantageous in biological applications since it facilitates desorption and ionization of large biomolecules in excess of 100,000 Da while keeping them intact MALDI has been successfully coupled with TOF mass spectrometers to analyze large molecules.

Ionization of the analyte is effected by pulsed laser radiation focused onto the probe tip which is located in a short (4 cm) source region containing an electric field. The molecular ions and/or fragment ions formed at the probe tip are accelerated by the electric field toward a detector through a flight tube, which is a long (4 m) field free drift region. Since all molecular ions receive the same amount of energy, the time required for ions to travel the length of the flight tube is dependent on their mass. Thus, low-mass ions have a shorter time of flight CTOF) than heavier ions. All the ions that reach the detector as the result of a single laser pulse produce a transient TOF signal. Typically, ten to several hundred transient TOF mass spectra are averaged to improve ion counting statistics.

The mass of an unknown analyte is determined by comparing its experimentally determined TOF to TOF signals obtained with ions of known mass. The MALDI-TOF-MS technique is capable of determining the mass of proteins of between 1 and 40 kDa with a typical accuracy of ±0.1%, and a somewhat lower accuracy for proteins of molecular mass above 40 kDa MALDI-TOF-MS has been used for the analysis of high molecular weight biomolecules (1 to 150 kDa) and synthetic polymers and to detect biological warfare agents in environmental air samples. Recent efforts have focused on the identification of bacteria using MALDI-TOF-MS to identify bacteria at the genus, species and strain levels

In addition to bacteria and bacterial spores, there are many other bioorganic compounds in the environment such as, for example, eukaryotic organisms (e.g., filamentous fungi, yeasts and molds), components of these organisms (e.g., spores and toxins produced by fungi) and pollen that contain molecules similar to those found in bacteria that could give rise to a false reading or confusion when analyzing a sample for the presence of bacteria. Previous techniques for identifying and classifying these bioorganic compounds include, for example, morphological examination of microscopic structures and biochemical tests. A problem associated with these techniques is that an improper preparation of the sample in the laboratory could lead to an inaccurate, if not impossible, identification of the organism.

Also, while the identification of bacteria has become somewhat routine, the identification of filamentous fungi is less defined than identifying bacteria. Fungi are much more closely related to one another than are bacteria and the complexity of these eukaryotic organisms makes it harder to identify them using advanced DNA analysis techniques such as, for example, Polymerase Chain Reaction (PCR). In addition, fungal DNA is more difficult to extract because of the reinforced cell wall (containing, e.g., chitin). Mechanical lysis (such as bead-beating) generally works better than chemical lysis protocols, but beating for too long will cause shearing of the cell. Even if one is able to extract and isolate DNA and develop appropriate protocols to utilize for routine analysis, the difficulty in identification comes from not having as complete a library database with which to compare obtained DNA for identification as are available for bacteria Similar problems exist in the identification of yeasts and molds.

Typical air samples collected from the environment are complex mixtures and may contain bacterial cells and spores, fungal spores and fragments, pollen grains, toxins produced by fungi, and other bioorganic compounds Hence, it is important to not only be able to identify the mass spectral signatures of threat agents such as bacteria, but also to characterize other bioorganic compounds in the background environment to reduce false positives in a risk assessment situation. Moreover, as the prior methods for identification and classification of filamentous fungi, yeasts, molds, toxins produced by fungi, and pollen are limited to morphological identification using microscopy and some basic biochemical tests and molecular methods, there exists a need for a more comprehensive and easier system by which these organisms and bioorganic compounds can be classified and identified.

Accordingly, there exists a need to develop a system thereby resulting in a more accurate and simplified method for the identification and rapid classification of filamentous fungi, yeasts, molds, toxins produced by fungi, and pollen

### SUMMARY OF THE INVENTION

Among other things, it is an object of the present disclosure to provide a method for the identification and classification of bioorganic compounds selected from the group consisting of filamentous fungi, yeasts, molds, toxins produced by fungi, and pollen grains employing mass spectrometry. It is an object to provide such a method for determining the identity of filamentous fungi, yeasts, molds, toxins produced by fungi, and pollen at the genus, species and strain levels. Moreover, in keeping with these and other objects of this disclosure, one analyzing a sample for the presence of biological warfare agents such as, for example, bacteria, bacterial spores and toxins, can utilize the present disclosure to identify these bioorganic compounds, which comprise background organic particles in the sample and could lead to false positives in a risk assessment situation.

In one embodiment of the present disclosure, a method is provided for generating biomarkers for filamentous fungi, yeasts, molds, toxins produced by fungi, and pollen at the genus, species or strain level employing mass spectrometry. It is also an objective to provide such a method that uses a MALDI-TOF mass spectrometer to generate these biomarkers. In another embodiment of the present disclosure, an unknown sample may be analyzed by mass spectrometry and spectra obtained for that sample may be compared with the foregoing biomarkers to quickly and easily identify the unknown sample. The present disclosure thus permits one to more quickly and easily identify and classify these bioorganic compounds than prior art methods.

Using the methods of the present disclosure, a library of biomarkers may advantageously be constructed from known samples to aid in future identification of filamentous fungi, yeasts, molds, toxins produced by fungi, and pollen. This library may be used in conjunction with other libraries of organisms such as, for example, bacteria, to accurately characterize the components of a sample from the environment.

The term “biomakerr” as used herein refers to an ion or charged molecular fragment produced by mass spectrometry that produces a unique peak and/or peaks on a mass spectrum at the genus, species and strain level.

The term “bioorganic compounds” as used herein refers to whole cells or cellular components of filamentous fungi, yeasts, molds or toxins of fungi, and pollen grams that will generate biomarkers when the cells or cellular components are subjected to mass spectrometry.

### BRIEF DESCRIPTION OF THE DRAWINGS

FIG. 1 is a picture of pollen grains of Ambrosia trifida (Giant Ragweed) taken through a Scanning Electron Microscope (SEM).

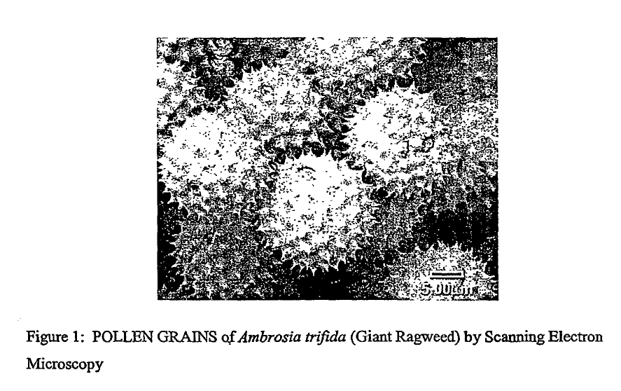

FIG. 2 are spectra obtained after MALDI-TOF-MS analysis on Juglans nigra in 4-HCCA, sinapinic acid (0.1% TFA), ferulic acid, and sinapinic acid (5% TFA) matrices.

FIG. 3 are spectra obtained after MALDI-TOF-MS analysis on Kochia scopartia in 4-HCCA, sinapinic acid (0.1% TFA), ferulic acid, and sinapinic acid (5% TFA) matrices.

FIG. 4 are spectra obtained after MALDI-TOF-MS analysis on Ambrosia trifida in 4HCCA, sinapinic acid (0.1% TFA), ferulic acid, and sinapinic acid (5% TFA) matrices.

FIG. 5 are spectra obtained after MALDI-TOF-MS analysis on Populus deltiodes in 4-HCCA, sinapinic acid (0.1% TFA), ferulic acid, and sinapinic acid (5% TFA) matrices.

FIG. 6 are spectra obtained after MALDI-TOF-MS analysis on Populus nigra italica in 4HCCA, sinapinic acid (0.1% TFA), ferulic acid, and sinapinic acid (5% TFA) matrices.

FIG. 7 are a group of spectra obtained after MALDI-TOF-MS analysis on pollen grains in ferulic acid matrix: the spectra are for (from bottom to top) Juglans nigra, Kochia scoparia, Ambrosia trifida, Populus deltiodes, and Populus nigra italica.

FIG. 8 are spectra obtained after MALDI-TOF-MS analysis on Juglans nigra and bacterial environmental isolates (Staphylococcus sp., Micrococcus sp., and Bacillus Sp.) in sinapinic acid.

FIG. 9 are spectra obtained after MALDI-TOF-MS analysis on a Penicillium sp., along with spectra of the matrix (sinapinic acid (SA)) and the agar upon which the Penicillium sp. was grown (Rose Bengal agar) as a control.

### DESCRIPTION OF THE PREFERRED EMBODIMENT

Methods for the identification and rapid classification of bioorganic compounds selected from the group consisting of filamentous fungi, yeasts, molds, toxins produced by fungi, and pollen grains at the genus, species and strain levels employing mass spectrometry, and the use of these compounds to generate individual biomarkers based on components of these compounds, are described hereinbelow. Accordingly, a library of the biomarkers may advantageously be constructed to aid in future identification of filamentous fungi, yeasts, molds, toxins produced by fungi, and pollen and, in turn, used in conjunction with other libraries of organisms such as, for example, bacteria, to accurately characterize and identify a pure sample or the components of a mixed sample. It is to be understood that the library can also be employed to characterize and identify components of a sample in a laboratory which, in turn, can be used in conjunction with other instruments or techniques, e.g., microscopy, PCR, biochemical testing via manual or automated systems, immunoassays, etc., to identify the bioorganic compounds.

In general, filamentous fungi for analyzing herein are eukaryotic organisms with multicellular structures bounded by a rigid cell wall containing, for example, chitin. The fungal cell wall is a structure that is subject to change and modification at different stages in the life of a fungus. The wall is ordinarily composed of at least a skeletal or microfibrillar component located on the inner side of the wall and usually embedded in all amorphous matrix material that extends to the outer surface of the wall. The skeletal component consists of at least some highly crystalline, water-insoluble materials that include, for example, beta-linked glucans and chitin, while the matrix consists mainly of polysaccharides, e.g., alpha-glucans, glycoproteins, etc., that are mostly water soluble. Additional components that may be present in the cell walls of fungi include, but are not limited to, lipids, melanins, D-galactosamine polymers, polyaronids, cellulose, sterol, ergosterol, etc. Fungi will ordinarily grow as multicellular colonies, e.g., mushrooms or molds, and form a mycellum through a mass of branching, interlocking filaments, or hyphae. Although these branches may be interrupted by cross-wails, the passage of cytoplasm between compartments is possible. Both sexual and asexual reproduction can occur in fungi. In asexual reproduction, spores, known as “conidia”, are borne externally at the tips of budding projections formed at various locations along the filaments. Most fungal spores range in size, e.g., from about 2 to about 50 μm. Spores and hyphal fragments of fungi are ubiquitous in air where they are sometimes the major pollutant and sources of infection or allergic reactions.

The families represented by the filamentous fungi herein include, but are not limited to, Phycomycetes, Ascomycetes, e.g., Neurospora, Aspergillus and Penicillium, Basidioniycetes and Deuteromycetes. In addition to species of these genera, filamentous fungi may be a cell of a species of, but not limited to, Acremonium spp., Altenaria spp., Arthinium spp., Aureobasidium spp., Beauveria spp., Bipolaris spp., Boryi gpp., Chaetomium spp., Chrysonilia spp., Cladosoporium spp., Cunninghamella spp., Cuwvularia spp., Drechskera spp., Emmonsia spp., Epiccoccum spp., Fiuarium spp., Humicola spp., Microsporun spp., Mucor spp., Myceliophthora spp., Paecilomyces spp., Pithomyces spp., Rhizomucor spp., Rhizopus spp., Scopulariopsis spp., Thielavia spp., Trchoderma spp., UMociadwum spp., Verticilliwn spp. and the like.

Yeasts and molds are known to those skilled in the art to be members of the fungal kingdom. Yeast for use herein are ordinarily a simple form of fungi that generally consist of single cells rather than hyphae. They typically reproduce asexually by budding (e.g., a small outgrowth on the cell's surface which increases in size until a wall forms to separate the new individual from the parent) and fission. Some yeast, such as, for example, Saccharomyces cerevisiae, (also referred to as brewer's yeast or baker's yeast) can exhibit, under certain conditions, a filamentous mold-like form. These filamentous cells, often referred to as pseudohyphal cells, have an elongated morphology.

Additionally, certain fungi, including molds, produce toxins such as T-2 mycotoxins and Aflatoxin B₁. In some instances, these toxins can be airborne.

Pollen herein are ordinarily in the form of grains which are microspores of seed plants containing, for example, a male gametophyte. Some pollen grains are dispersed by the wind and may be allergenic to pollen-sensitive individuals. Common pollen grains include those produced by grasses, trees, shrubs and weeds, and include species of; among others, Sorghum spp., Secale spp., Poa spp., Cynodon spp., Dactylis spp., Agrostis spp., Zea spp., Ulmus sp., Juglans spp., Populus spp., Juniperus spp., Fraxinus spp., Betula spp., Alnus spp., Acer spp., Kochia spp., Iva spp., Artemista spp., and Ambrosia spp. Pollen can also include, for example, proteins, polypeptides, polysaccharides, glycoproteins, and lipoproteins in both the pollen outer wall and cytoplasm.

The foregoing filamentous fungi, yeasts, molds, toxins produced by fungi, and pollen can be analyzed in the form of, for example, an air sample, lab sample, etc., employing known commercially available mass spectrometers. Suitable mass spectrometers for use herein include, but are not limited to, linear or non-linear reflectron time-of-fight, single or multiple quadrupole, single or multiple magnetic sector, Fourier Transform ion cyclotron resonance (FTICR), ion trap, and combinations thereof (e.g., ion-trap/time-of-flight). Typically, the analysis using mass spectrometry subjects the sample to an ionization source employing commercially available ionization formats which include, but are not limited to, laser desorption methods (including MALDI), FAB, plasma desorption, continuous or pulsed ESI and related methods (e.g., Ionspray or Thermospray), or MCI to produce charged molecular ions. These ions are then propelled into a mass analyzer to obtain a mass spectra.

In a preferred embodiment MALI-TOF-MS is the mass spectrometer utilized to classify and identify the bioorganic compounds. Relative to gas or liquid chromatographic systems employing mass spectrometric detection, sample preparation is relatively minimal because MALDI-TOF-MS is tolerant of buffers, salts, and many other contaminants. As a result, each analysis requires only a few seconds to perform. Under ideal conditions, spectra can be obtained from femto- and attomole quantities of analyte. In addition to providing molecular weight information, the amino-acid sequence of unpurified peptides of less than about 3 kDa can be determined using post-source decay and collision-induced dissociation methods.

In general, a matrix solution is first prepared for addition to an unknown sample. The matrix advantageously transfers energy nondestructively from the laser beam to the sample, as discussed hereinbelow, thereby producing intact, large molecular ions in the gas phase. The matrix is ordinarily formed by combining, for example, a suitable organic acid with an aqueous solvent to form the matrix solution to which the unknown sample that may contain one or more of the foregoing bioorganic compounds is added. Suitable organic acids for use herein include, but are not limited to, low molecular weight aromatic organic acids such as, for example, 2,5-dihydroxybenzoic acid, α-cyano-4-hydroxycinnamic acid (4 HCCA), 3,5-dimethoxy-4-hydroxycinnamic acid (sinapinic acid), transhydroxy-3-methoxycinanic acid (ferulic acid) and the like and mixtures thereof. Suitable solvents for use herein in forming the matrix solution include, but are not limited to, organic solvents such as nitrites, e.g., acetonitrile; alcohols such as mono- or polyfunctional, unsubstituted or substituted, aliphatic alcohols, e.g., lower alkanols such as methanol ethanol, propanol, etc.; ethers such as tetrahydrofuran, dioxane and diethyl ether, and the like; water and the like and mixtures thereof. Generally, the matrix solution will be formed by adding the organic acid and solvent in various ratios, including, for example, ranging from about 70/30 (v/v) to about 30/70 (v/v) or in a ratio of 50/50 (v/v). If desired, additional components can be added to the matrix solution to assist in ionization of the analyte such as, e.g., trifluoroacetic acid (TFA). Amounts of these components can vary, ranging from about 0.1% to about 5% or more.

Once the matrix solution is prepared, the unknown sample which may contain one or more of the foregoing bioorganic compounds is placed on a metal probe, mixed with the foregoing matrix solution and allowed to dry by techniques known in the art, e.g., air-dried at room temperature, placed in a vacuum, etc. The unknown sample is ordinarily placed on the probe as a solution and mixed with an excess amount of prepared matrix solution. However, as one skilled in the art would readily appreciate, the order in which the sample and matrix solution are placed on the probe and mixed can vary. For example, the sample can first be placed on the probe, matrix solution added and mixed with the sample and then allowed to dry, or the matrix solution can be placed on the probe, sample solution added and mixed with the matrix solution and then allowed to dry. Alternatively, a portion of the matrix solution can be placed on the probe, then the sample solution can be added, followed by the additional of the remaining portion of the matrix solution and the mixture then allowed to dry.

The sample/matrix mixture is then subjected to MS analysis in a MALDI-TOF instrument equipped with at least a nitrogen laser, e.g., the analyte in matrix solution is subjected to irradiation with a pulsed UV or IR laser beam in a vacuum chamber. The laser shot produces desorption and ionization of matrix and analyte with resulting charged molecular ions. A complete spectrum can be acquired with one laser shot, however, it is more advantageous to obtain and average several hundred laser shots. In one embodiment, about 10 to about 500 laser shots are obtained and averaged. In a preferred embodiment, about 10 to about 100 laser shots are obtained and averaged. In a most preferred embodiment, a 337 nm nitrogen laser is used with about 50 laser shots averaged. Generally, the mass spectra are averaged over one of the foregoing ranges of laser shots and compared to known MALDI-TOF-MS spectra of bioorganic compounds to identify the unknown sample.

As stated above, the present disclosure provides at least a method for generating unique mass spectra for filamentous fungi, yeasts, molds, toxins produced by fungi, and pollen grains. These profiles contain biomarkers which distinguish between filamentous fungi, yeasts, molds, toxins produced by fungi, and pollen of different genera, species and strains. Comparable profiles are generated when the method is performed using different mass spectrometry techniques and different MALDI-TOF instruments from different manufacturers.

The capability for rapid identification of these bioorganic compounds, including filamentous fungi, yeasts, molds, toxins produced by fungi, pollen, etc., using MALDI-TOF-MS has been demonstrated. Reproducible spectra were obtained not only between replicate samples but also across different matrix/solvent preparations. In addition, biomarker ions, or charged molecular ions or fragment ions, were obtained with good signal-to-noise ratios and resolution. These biomarkers permit quicker and easier identification of these bioorganic compounds than prior art methods, which require morphological examination by light microscopy or biochemical testing, etc.

Known biomarkers may be used to construct libraries to allow one to more rapidly and easily identify the bioorganic compounds present in a sample. In addition, rapid identification of fungi, yeast, mold, toxins produced by fungi, and pollen grains via MAILDI-TOF-MS has potential applications in environmental analyses of air for detection of allergens and also in the field of detection of biological warfare agents. Major charged molecular fragments observed from these bioorganic compounds make them easily distinguishable from the mass spectra of other microorganisms found in the environment, such as bacteria, and those especially considered to be biological warfare threat agents. I this respect, it is feasible to identify bioorganic compounds which add to the background of a measurement of a complex environmental air sample in order to accurately identify those that pose a health risk versus those that do not.

The following non-limiting examples are illustrative of the methods using a MALDI-TOF-MS for the identification and characterization of these bioorganic compounds in accordance with the present disclosure.

### EXAMPLES

Materials and Methods

Solvent components for the matrix solutions were obtained from J. T. Baker (Phillipsburg, N.J., USA). These included acetonitrile (ACN, HPLC grade), water (HPLC grade), and trifluoroacetic acid (IFA). MALDI matrices used for this study were purchased from Aldrich (Milwaukee, Wis., USA) and included 3,5-dimethoxy-4-hydroxycinnamic acid (sinapinic acid), α-cyano 4-hydroxycinnamic acid (4HCCA), and trans-4-hydroxy-3-methoxycinnamic acid (ferulic acid). All matrices were supplied and used without further purification. The matrix solutions employed in this study are set forth below in Table 1.

TABLE 1
Matrix Solution

| Organic Acid | Solvent |
|---|---|
| 3,5-dimethoxy-4-hydroxycinnamic acid | 70/30 (v/v) ACN/H₂O with |
| | 0.1% TFA |
| 3,5-dimethoxy-4-hydroxycinnamic acid | 70/30 (v/v) ACN/H₂O with |
| | 5% TFA |
| α-cyano-4-hydroxycinnamic acid | 70/30 (v/v) ACN/H₂O with |
| | 0.1% TFA |
| trans-4-hydroxy-3-methoxycinnamic | 30/70 (v/v) ACN/H₂O with |
| acid | 0.1% TFA |

Calibration standards were purchased from Sigma (St Louis, Mo., USA) and included Cytochrome C and Angiotensin II. Calibration standard solutions were prepared in water with 0.1% TFA.

MALDI-TOF mass spectra were obtained using a Kratos Kompact MALDIIV mass spectrometer (Manchester, UK) in linear, positive ion mode. A 337 nm nitrogen laser was used with 50 laser shots averaged over a single well of a Kratos 20-well slide.

External mass calibrations were performed using Cytochrome C and Angiotensin II, calibrating on the molecular ion ([MH]⁺) of Angiotensin II and the molecular and doubly charged ([M+2H]₂₊) ions of Cytochrome C. A three-point calibration was applied in order to more accurately cover the broad mass range of peaks produced in the spectra.

### Example 1

The following example reports on the investigation of several species of pollen with the intention of obtaining biomarker signatures useful in distinguishing them from the mass spectral signatures of other microorganisms that can be found in an environmental air sample. Data is also presented that potentially may be used to differentiate between pollen species.

Weed and tree pollen were analyzed directly via MALDI-TOF-MS, with minimal sample preparation in a variety of matrix/solvent combinations. The analyses resulted in unique, complex mass spectrometric fingerprints (with ion masses in the range of 1 kDa to 12 kDa) of different species of intact pollen grains. The observed biomarkers enabled detection of the pollen grains and also distinguished them from the mass spectral signatures of other background environmental organisms and other pollen grains.

Pollen grains analyzed for this study were obtained from Sigma (St. Louis, Mo.) and included: Juglans nigra (Black Walnut), Kochia scoparia (Firebush), Ambrosia trifida (Giant Ragweed), Populus deltiodes (Eastern Cottonwood), and Populus nigra italica (Lombardy Poplar).

Pollen grains were suspended in filter sterilized water containing 0.1% TFA to a final concentration of 5 mg/mL and vortexed. A 0.5 μL aliquot of each pollen solution was deposited into a well of the stainless steel sample slide followed by the addition of 0.5 μL of one of the four matrix solutions discussed above. Samples were allowed to dry at room temperature and then inserted into the mass spectrometer for analysis.

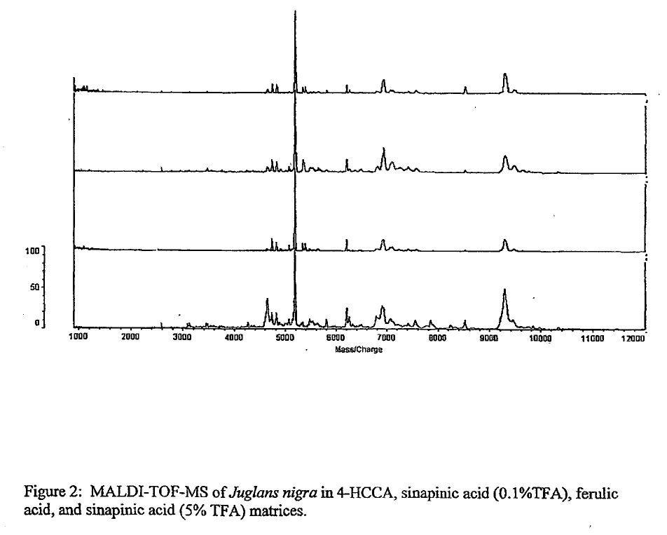

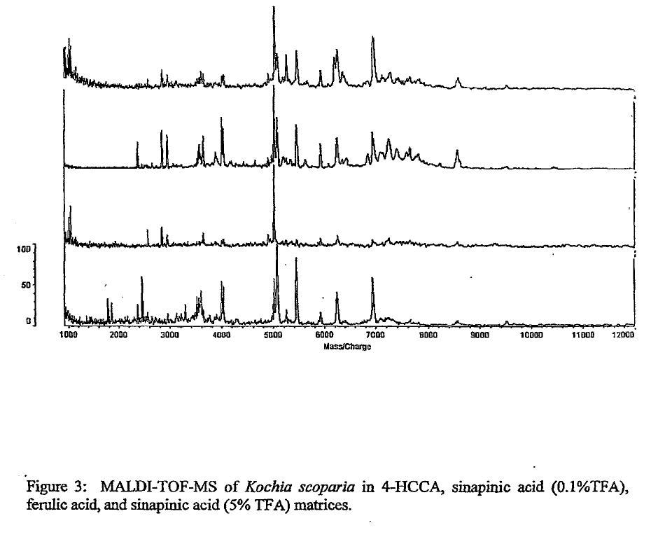

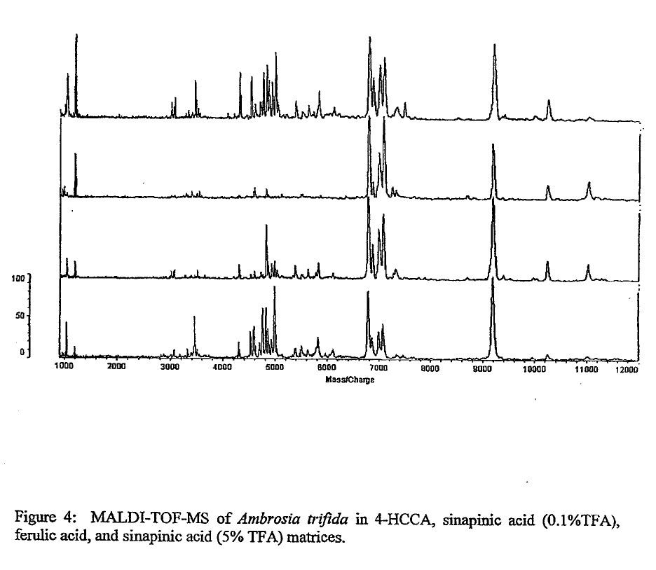

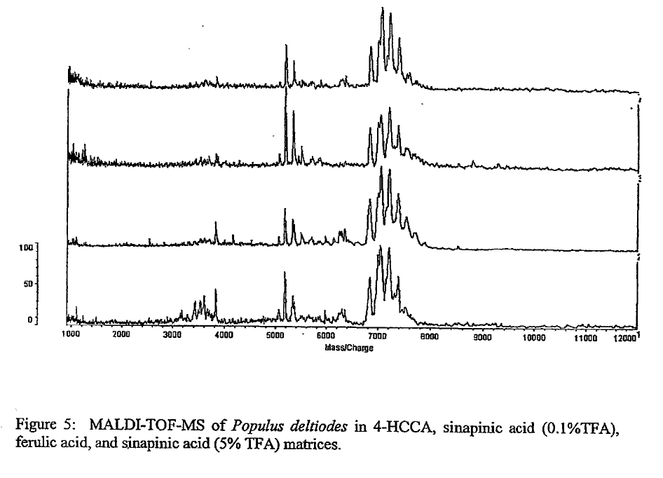

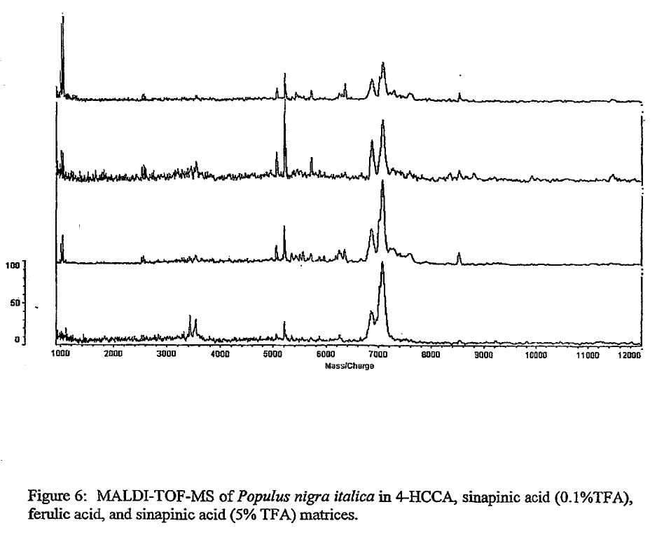

Mass spectra were obtained directly from intact pollen grains. The representative MALDI-TOF mass spectra for each of the pollens studied in this sample are shown in FIGS. 2 through 6. The figures, one for each individual pollen species, show qualitatively similar spectral patterns in the mass range between 1 to 12 kD with little spectral variation observed between the different matrix/solvent combinations. The minor variations (e.g., slight mass shifts) observed may be due to sample-to-sample differences in surface morphology (sample thickness, roughness, homogeneity, etc.) or morphology of the pollen grain itself pollen grains range in size from 5 to 200 μm).

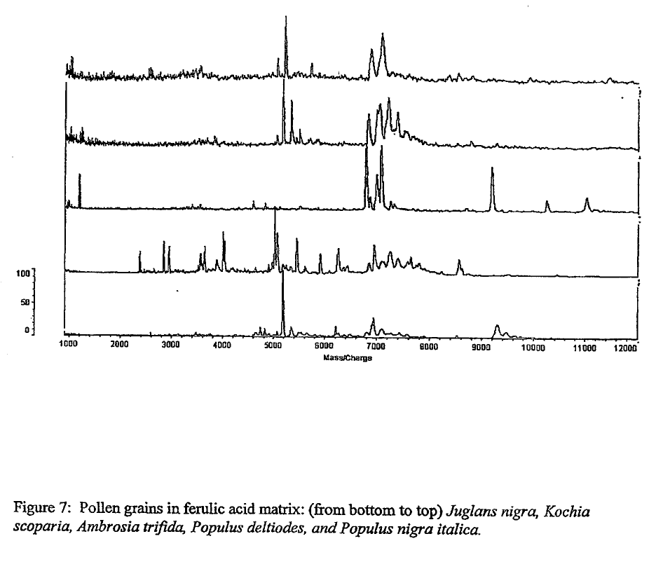

FIG. 7 presents all five of the pollen species in a single matrix, ferulic acid. It can be seen from this figure that although some of the peaks are common to several of the pollen species studied, there are unique spectral features for each species. These unique peaks correspond to biomarkers that allow one to distinguish between pollen at the genus, species and strain levels.

### Example 2

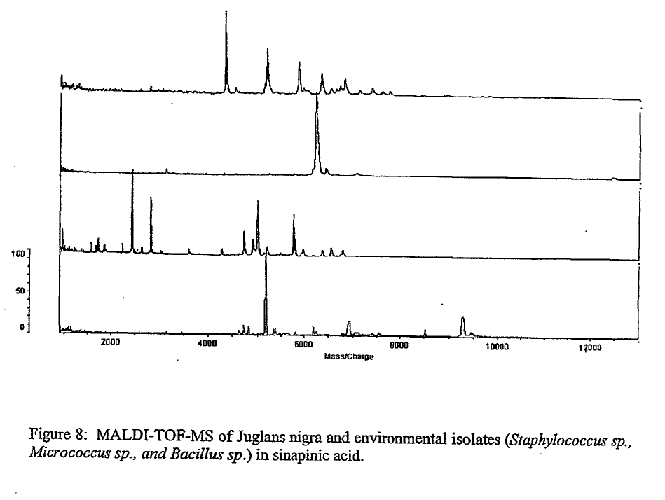

In an effort to illustrate the utility of MAILDI-TOF-MS for differentiating between the mass spectra of background environmental organisms such as bacteria and pollen, MALDI-TOF-MS was run on three bacteria (Staphylococcus sp., Micrococcus sp., and Bacillus sp.) and pollen grains of Juglans nigra. MALDI-TOF-MS was performed as described above in Example 1. FIG. 8 presents the spectra of the three bacteria and the pollen grains of Juglans nigra. From this figure, it can be seen that pollen produces a mass spectral fingerprint that can be easily distinguished from the fingerprints of the bacteria In addition, using MALDI-TOF-MS to analyze pollen is advantageous because, unlike bacteria which undergo significant changes in their bacterial protein expression during the phases of growth (i.e., lag, log, exponential, and stationary), pollen do not exhibit such dynamic changes. Unlike bacteria, pollen also do not display major changes due to environmental factors. This could explain the excellent reproducibility between replicate samples of the same pollen preparations in the different matrix/solvent combinations.

### Example 3

A method to aid in the detection and identification of filamentous fungi via MALDI-TOF-MS was demonstrated. Penicillium spp. were analyzed directly via MALDI-TOF-MS following the procedures described above in Example 1 except the spores and hyphal fragments were removed and suspended in water with minimal sample preparation. The analysis resulted in a unique, complex mass spectrometric fingerprint. The observed biomrarkers enable detection and specification of the Penicillium spp. and distinguish them from the mass spectral signatures of other background environmental organisms.

Penicillium spp. analyzed for this study were cultured from the environment using as a collector (1) a New Brunswick Slit Sampler with Trypticase Soy Agar or Saboraud Dextrose Agar and (2) a Millipore MairT Collector with Saboraud Dextrose Agar or Rose Bengal Agar. Subsequently, the fungi were transferred to Potato Dextrose Agar for culture at room temperature for 5-7 days.

Fungal spores, hyphal fragments, etc. were removed from the surface of the agar plate and transferred to a tube containing sterile water with 0.1% TFA and vortexed. A 0.5 μL aliquot of each fungal solution was deposited into a well of the stainless steel sample slide followed by the addition of 0.5 μL of sinapinic acid matrix Samples were allowed to dry and were then inserted into the mass spectrometer for analysis.

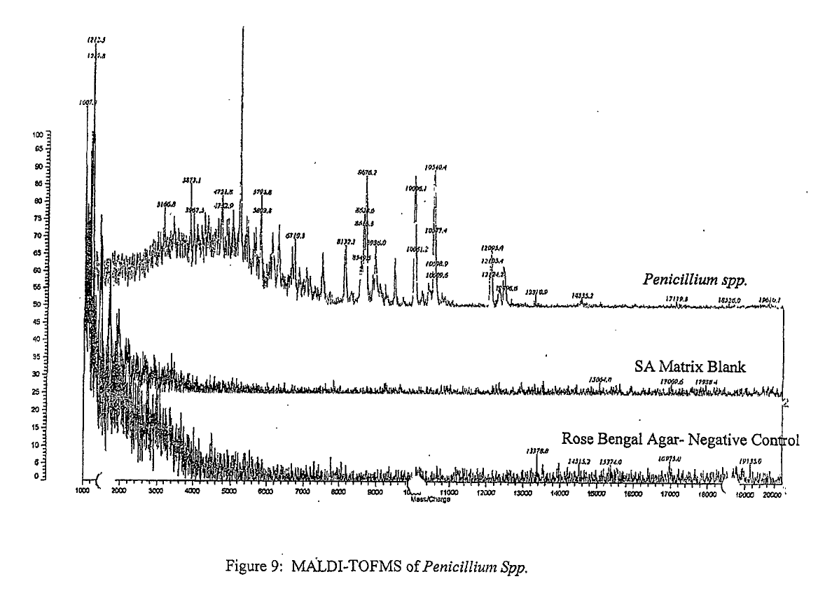

The representative MALDI-TOF mass spectra for a Penicillium sp. is shown in FIG. 9, along with spectra of the matrix (sinapinic acid (SA)) and the agar upon which the Penicillium sp. was grown (Rose Bengal agar) as controls. It can be seen from this figure that the Penicillium sp. produces it own unique mass spectrometric signature.

It will be understood that various modifications may be made to the embodiments disclosed herein. Therefore the above description should not be construed as limiting, but merely as exemplifications of preferred embodiments. For example, different types of filamentous fungi, yeasts, molds, toxins produced by fungi, and pollen other than those disclosed herein can be analyzed and biomarkers generated therefrom Those skilled in the art will envision other modifications within the scope and spirit of the claims appended hereto.

What is claimed is:

1. A method for generating biomarkers specific for a known genus, species, or strain of a bioorganic compound selected from the group consisting of filamentous fungi, yeasts, molds, toxins of fungi, and pollen comprising:
(a) providing a sample comprising a known genus, species or strain of the bioorganic A 5 compound;
(b) placing an aliquot of said sample into a mass spectrometer;
(c) subjecting the sample to an ion source to produce charged molecular ions;
(d) propelling the ions into a mass analyzer to obtain a mass spectra;
(e) repeating steps (a)-(d) with at least one other non-identical sample comprising the same genus, species or strain of bioorganic compound;
(f) comparing the mass spectra obtained for each sample;
(g) identifying at least one peak on the spectra that is common to each sample; and
(h) assigning an m/z measurement of the peak as a genus, species, or stain specific biomarker.

2. The method according to claim 1 wherein the mass spectrometer is selected from the group consisting of linear or non-linear reflectron time-of-flight, single or multiple quadrupole, single or multiple magnetic sector, fourier transform ion cyclotron resonance, ion trap and combinations thereof.

3. The method according to claim 1 wherein the ion source is selected from the group consisting of laser desorption, fast atom bombardment, plasma desorption, electrospray ionization, or massive cluster impact.

4. The method according to claim 1 wherein the mass spectrometer is a time-of-flight mass spectrometer.

5. The method according to claim 4 wherein matrix assisted laser desorption ionization is used as the ion source.

6. The method according to claim 5 comprising the steps of:
(a) mixing a sample comprising a suspension of known genus, species or strain of the bioorganic compound with a matrix solution to generate a sample mixture;
(b) placing the aliquot of said sample mixture on the probe tip of the time-of-flight mass spectrometer and allowing it to dry;
(c) irradiating the dried aliquot with pulsed laser radiation to form charged molecular ions;
(d) accelerating the charged molecular ions by an electric field toward a detector through the flight tube of the time-of-flight mass spectrometer to obtain a mass spectra;
(e) averaging the mass spectra resulting from 10 to 500 laser pulses;
(f) repeating steps (a)-(e) with at least one other, nonidentical bioorganic compound comprising a suspension of the same genus, species or strain;
(g) comparing the averaged mass spectra obtained for each bioorganic compound;
(h) identifying at least one peak that is common to each bioorganic compound; and
(i) assigning an m/z measurement of the peak as a genus, species, or strain specific biomarker.

7. The method of claim 6 wherein the matrix solution comprises one or more organic acids in an aqueous solvent solution.

8. The method of claim 7 wherein the organic acids are selected from the group consisting of 3,5-dimethoxy-4-hydroxycinnamic acid, ∀-cyano-4-hydroxycinnamic acid and trans-4-hydroxy-3-methoxycinnamic acid.

9. The method of claim 7 wherein the aqueous solvent solution is an organic solvent selected from the group consisting of nitrites, alcohols, ethers, water and mixtures thereof.

10. The method of claim 7 wherein the organic acids are selected from the group consisting of 3,5-dimethoxy-4-hydroxycinnamic acid, ∀-cyano-4-hydroxycinnamic acid and trans-4-hydroxy-3-methoxycinnamic acid and the aqueous solvent solution is an organic solvent selected from the group consisting of acetonitrile, alcohols, water and mixtures thereof.

11. The method of claim 7 wherein the matrix solution further comprises aqueous trifluoroacetic acid.

12. The method of claim 10 wherein the organic acid and organic solvent are added in a ratio from about 70/30 (v/v) to about 30/70 (v/v).

13. The method of claim 6 wherein the pulsed laser radiation is provided by a 337 nm nitrogen laser.

14. The method of claim 6 wherein about 10 to about 100 spectra are averaged.

15. A method for determining the genus, species and/or stain of an unknown bioorganic compound which comprises:
(a) generating a mass spectrum of the unknown bioorganic compound according to steps (a)-(d) of claim 1; and
(b) comparing the mass spectrum of the unknown bioorganic compound to a plurality of genus, species or strain specific biomarkers, said biomarkers being generated according to claim 1.

16. A method for determining the genus, species and/or strain of an unknown bioorganic compound which comprises:
(a) generating a mass spectrum of the unknown bioorganic compound according to steps (a)-(e) of claim 6; and
(b) comparing the averaged mass spectrum of the unknown bioorganic compound to a plurality of genus, species or strain specific biomarkers, said biomarkers being generated according to claim 6.

17. The method of claim 16 wherein the matrix solution comprises one or more organic acids in an aqueous solvent solution.

18. The method of claim 17 wherein the organic acids are selected from the group consisting of 3,5-dimethoxy-4-hydroxycinnamic acid, V-cyano-4-hydroxycinnamic acid and trans-4-hydroxy-3-methoxycinnamic acid and the aqueous solvent solution is an organic solvent selected from the group consisting of nitrites, alcohols, ethers, water and mixtures thereof.

19. The method of claim 18 wherein the matrix solution further comprises aqueous trifluoroacetic acid.

20. The method of claim 16 wherein the pulsed laser radiation is provided by a 337 nm nitrogen laser.

21. The method of claim 16 wherein about 10 to about 100 spectra are averaged.

22. A biomarker library for identifying the genus, species and/or strain of an unknown bioorganic compound selected from the group consisting of filamentous fungi, yeasts, molds, toxins of fungi, and pollen, the library comprising genus, species or strain specific biomarkers for known bioorganic compounds generated by the method of claim 1.

23. A biomarker library for identifying the genus, species and/or strain of an unknown bioorganic compound selected from the group consisting of filamentous fungi, yeasts, molds, toxins of fungi, and pollen, the library comprising genus, species or strain specific biomarkers for known bioorganic compounds generated by the method of claim 6.

24. The library of claim 22 wherein the genus, species and/or strain of fungi used is selected from the group consisting of Phycomycetes, Ascomycetes, Neurospora, Aspergillus, Penicillium, Basidiomycetes, Deuteromycetes, Aeremonium spp., Alternaria spp., Arthrinium spp., Aureobasidium spp., Beauveria spp., Bipolaris spp., Boryts spp., Chaetomium spp., Chirysonilia spp., Cladosoporium spp., Cunninghamella spp., Curvularia spp., Drechslera spp., Emmonsia spp., Epiccoccum spp., Fusarium spp., Humicola spp., Microsporum spp., Mucor spp., Myceliophthora spp., Paecilomyces spp., Pithomyces spp., Rhizomucor spp., Rhizopus spp., Scopulariopsis spp., Thielavia spp., Trichoderma spp., Ulocladium spp. and Verticillium spp.

25. The library of claim 22 wherein the pollen used is selected from the group consisting of Sorghum spp., Secale spp., Poa spp., Cynodon spp., Dactylis spp., Agrostis spp., Zea spp., Ulmus spp., Juglans spp., Populus spp., Juniperus spp., Fraxinus spp., Betula spp., Alnus spp., Acer spp., Kochia spp., Iva spp., Artemisia spp., and Ambrosia spp.
